(19) United States
(12) Patent Application Publication
(10) Pub. No.: US 2004/0209916 A1
(43) Pub. Date: Oct. 21, 2004

(54) Combination therapies

(75) Inventors: Ivan Michael Richards, Kalamazoo, MI (US); Robert Everett Manning, St. Louis, MO (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/824,315

(22) Filed: Apr. 13, 2004

Related U.S. Application Data
(60) Provisional application No. 60/463,975, filed on Apr. 18, 2003.

(51) Int. Cl. A61K 31/55; A61K 31/445; A61K 31/40; A61K 31/235; A61K 31/195

(52) U.S. Cl. 514/317; 514/408; 514/567; 514/532; 514/643; 514/554

(57) ABSTRACT

The invention is directed to methods of treating asthma, COPD, allergic rhinitis, and infectious rhinitis by administering a first pharmaceutical agent including one or more compounds selected from the quarternary ammonium compounds of formulae I-V and a second pharmaceutical agent including one or more pharmaceutical agents selected from Adenosine A₂ₐ Receptor Agonists, D2-Dopamine Receptor Agonists, Phosphodiesterase Inhibitors (PDE's), corticosteroids, norepinephrine reuptake inhibitors, 4-hydroxy-7-[2-[2-[3-[2-phenylethoxy]-propylsulphonyl]ethylamino]ethyl]-1,3-benzothiazol-2(3H)-one, and pharmaceutically acceptable salts thereof, and non-quarternized antimuscarinic compounds.

### CROSS REFERENCE TO RELATED APPLICATIONS

This application claims the benefit of the following provisional application: Application Ser. No. 60/453,975 filed Apr. 18, 2003 under 35 U.S.C. 119(e)(1).

### TECHNICAL FIELD

The present invention concerns a method for the treatment of asthma, a group of breathing disorders termed Chronic Obstructive Pulmonary Disease (COPD), allergic rhinitis, and infectious rhinitis.

### BACKGROUND OF THE INVENTION

“Asthma” refers to a chronic lung disease causing bronchoconstriction (narrowing of the airways) due to inflammation (swelling) and tightening of the muscles around the airways. The inflammation also causes an increase in mucus production, which causes coughing that may continue for extended periods. Asthma is generally characterized by recurrent episodes of breathlessness, wheezing, coughing, and chest tightness, termed exacerbations. The severity of exacerbations can range from mild to life threatening. The exacerbations can be a result of exposure to e.g. respiratory infections, dust, mold, pollen, cold air, exercise, stress, tobacco smoke, and air pollutants.

“COPD” refers to Chronic Obstructive Pulmonary Disease, primarily associated with past and present cigarette smoking. It involves airflow obstruction, mainly associated with emphysema and chronic bronchitis. Emphysema causes irreversible lung damage by weakening and breaking the air sacs within the lungs. Chronic Bronchitis is an inflammatory disease, which increases mucus in the airways and bacterial infections in the bronchial tubes, resulting in obstructed airflow.

“Allergic rhinitis” refers to acute rhinitis or nasal rhinitis, including hay fever. It is caused by allergens such as pollen or dust. It may produce sneezing, congestion, runny nose, and itchiness in the nose, throat, eyes, and ears.

“Infectious rhinitis” refers to acute rhinitis or nasal rhinitis of infectious origin. It is caused by upper respiratory tract infection by infectious rhinoviruses, coronaviruses, influenza viruses, parainfluenza viruses, respiratory syncytical virus, adenoviruses, coxsackieviruses, echoviruses, or Group A beta-hemolytic Streptococci and generically referred to as the common cold. It may produce sneezing, congestion, runny nose, and itchiness in the nose, throat, eyes, and ears.

### SUMMARY

In general, the invention features a method of treating asthma, COPD, allergic rhinitis, and infectious rhinitis by administering a first pharmaceutical agent including one or more compounds selected from the quarternary ammonium compounds of formulae I-V and a second pharmaceutical agent including one or more pharmaceutical agents selected from Adenosine A₂ₐ Receptor Agonists, D2-Dopamine Receptor Agonists, Phosphodiesterase Inhibitors (PDE's), corticosteroids, norepinephrine reuptake inhibitors, 4-hydroxy-7-[2-[2-[3-[2-phenylethoxy]-propylsulphonyl]ethylamino]ethyl]-1,3-benzothiazol-2(3H)-one, and pharmaceutically acceptable salts thereof, and non-quarternized antimuscarinic compounds.

The first pharmaceutical agent comprises compounds of the formulae I-V described below:

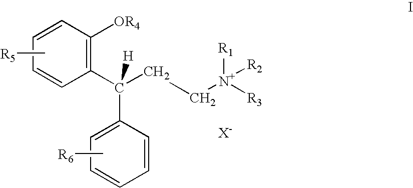

and the enantiomer thereof

wherein each R₁, R₂, and R₃ is independently H, C₁-C₅ alkyl optionally substituted with phenyl, or C₂-C₆ alkenyl, or wherein two of R₁, R₂ and R₃ may form a ring together with the quaternary ammonium nitrogen.

where R₄ is

—H,

—CO—R₄₋₁ where R₄₋₁ is

C₁-C₄ alkyl,

C₁-C₄ alkoxy,

—NR₄₋₂R₄₋₃ where R₄₋₂ and R₄₋₃ are the same or different and are —H or C₁-C₄ alkyl,

where R₅ and R₆ are the same or different and are

—H,

C₁-C₄ alkyl optionally substituted with 1 or 2

—OH,

C₁-C₄ alkoxy,

—COOH,

—CO—O—(C₁-C₃ alkoxy)

—F, —Cl, Br,

—CF₃,

where X⁻ is selected from the group consisting of the anions of the following acids hydrochloric acid; hydrobromic acid; hydroiodic acid; sulfuric acid; phosphoric acid; nitric acid; citric acid; methanesulfonic acid; CH₃—(CH₂)ₙ—COOH, where n is 0 to 4; HOOC—(CH₂)ₘ—COOH, where m is 1 to 4; HOOC—CH═CH—COOH; or benzoic acid;

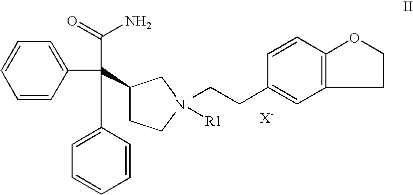

and any stereoisomers thereof, wherein

R₁ is selected from C₁-C₆ alkyl, —CH₂—(C₁-C₄ alkenyl), and —CH₂-(C₁-C₆ alkynyl), each of which is optionally substituted with a group selected from phenyl, C₁-C₄ alkoxy, and hydroxyl; and

X represents an anion of a pharmaceutically acceptable acid.

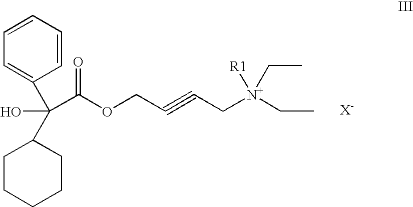

and any stereoisomers thereof, wherein

R₁ is selected from C₁-C₆ alkyl, —CH₂-(C₁-C₄ alkenyl), and —CH₂-(C₁-C₆ alkynyl), each of which is optionally substituted with a group selected from phenyl, C₁-C₄ alkoxy, and hydroxyl; and

X represents an anion of a pharmaceutically acceptable acid.

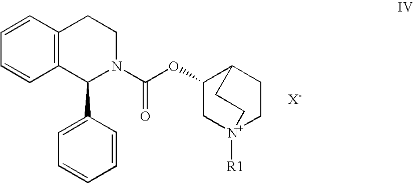

and any stereoisomers thereof, wherein

R₁ is selected from C₁-C₆ alkyl, —CH₂-(C₁-C₄ alkenyl), and —CH₂—(C₁-C₆ alkynyl), each of which is optionally substituted with a group selected from phenyl, C₁-C₄ alkoxy, and hydroxyl; and

X represents an anion of a pharmaceutically acceptable acid.

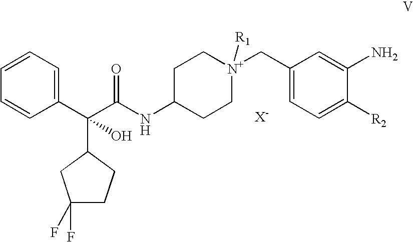

and any stereoisomers thereof, wherein

R₁ is selected from C₁-C₆ alkyl, —CH₂-(C₁-C₄ alkenyl), and —CH₂-(C₁-C₆ alkynyl), each of which is optionally substituted with a group selected from phenyl, C₁-C₄ alkoxy, and hydroxyl;

R₂ is selected from H or OH; and

X represents an anion of a pharmaceutically acceptable acid.

Embodiments of the invention may include one or more of the following. X⁻ is selected from the group consisting of the anions of the following acids: tartaric, hydrochloric, hydrobromic, hydroiodic, sulfuric, phosphoric, nitric, citric, methanesulfonic, CH₃—(CH₂)ₙ—COOH where n is 0-4, HOOC—(CH₂)ₘ—COOH where m is 1-4, HOOC—CH═CH—COOH, and benzoic. Preferably, X⁻ is selected from the group consisting of iodide, bromide, and chloride.

In describing embodiments, certain terminology will be utilized for the sake of clarity. Such terminology is intended to encompass the recited embodiments, as well as all technical equivalents that operate in a similar manner for a similar purpose to achieve a similar result. To the extent that any pharmaceutically active compound is disclosed or claimed, it is expressly intended to include all active metabolites produced in vivo, and, is expressly intended to include all enantiomers, isomers or tautomers where the compound is capable of being present in its enantiomeric, isomeric or tautomeric form. All stereoisomers have useful activity. Therefore, the invention includes use of each stereoisomer separately, as well as mixtures thereof.

### DESCRIPTION OF THE INVENTION

In general, the invention features a method of treating asthma, COPD, allergic rhinitis, and infectious rhinitis by administering a first pharmaceutical agent and a second pharmaceutical agent.

The first pharmaceutical agent includes one or more quarternary ammonium compounds of the formulae I-V.

The compounds of formulae I-V can be prepared by one skilled in the art. The quaternary ammonium compounds of formulae I-V may be prepared by means, well known to those skilled in the art, for preparing quaternary ammonium compounds from tertiary amines. For instance, the quaternary ammonium compounds may be produced by alkylating the tertiary nitrogen using the tertiary amines of U.S. Pat. Nos. 5,096,890, 5,973,182, 5,382,600, WO98/29402, of European Patent No. 0801067 A1, U.S. Patent Application No. 2001/0051727A1, and U.S. Pat. No. 5,382,600, the contents of which are hereby incorporated by reference, and other known compounds as starting materials.

The general term “quaternary ammonium compound” relates to any compound that can be regarded as derived from ammonium hydroxide or an ammonium salt by replacement of all four hydrogen atoms of the NH₄-ion by organic groups. The specific compounds are for nomenclature reasons (see e.g. Chemical Abstracts) named as “aminium” compounds, but it is possible to use the term “ammonium” in the names. For example, (3R)-3-(2-hydroxy-s-methylphenyl)-N,N-diisopropyl-N-methyl3phenylpropanl. aminium bromide can also be named as an ammonium compound: (3R)-[3-(2-hydroxy-s-methylphenyl)-3-phenylpropyl]diisopropylmethylammonium bromide.

By way of example, a tertiary amine according to U.S. Pat. No. 5,096,890, or its salt, is dissolved in a suitable solvent. The tertiary amine is allowed to react with an organic substrate, e.g. an organic halide. The substrate contains a C₁-C₆ alkyl, preferably a C₁-C₃ alkyl, optionally substituted with phenyl, and a leaving group. The identity of the leaving group is not critical, but it is preferred that the leaving group is a halide, such as iodide or bromide. Thus, exemplary substrates include methyl iodide, methyl bromide, ethyl iodide, propyl iodide, benzyl bromide or benzyl iodide. The resulting reaction product is a quaternary ammonium compound, which is readily crystallized in suitable solvents, known to those skilled in the art. The crystals thus produced are quaternary ammonium salts. Their identity is confirmed by standard methods, such as melting point determination, nuclear magnetic resonance (NMR) analysis and mass spectrometry.

The compounds of the invention are preferably administered as quarternary ammonium salts which include counter ions. X represents the anion, e.g., the counter ion, of a pharmaceutically acceptable acid. For instance X may be selected from the following anions: tartrate, chloride, bromide, iodide, sulfate, phosphate(s), nitrate, citrate, methanesulfonate, carboxylates with from two to six carbon atoms, dicarboxylates with from two to six carbon atoms, maleate, fumarate, and benzoate. For other acceptable quarternary ammonium salts, see Int. J. Pharm., 33, 201-217 (1986). Particularly preferred ions are chloride, iodide and bromide, especially bromide and iodide.

The substituent R₁ is selected from the group including C₁-C₆ alkyl, straight or branched, optionally substituted with 1-2 of phenyl or hydroxyl, or both. Thus, R₁ independently represent methyl, ethyl, propyl, isopropyl, butyl, isobutyl, pentyl, isopentyl, hexyl, or isohexyl, optionally substituted with 1-2 of phenyl or hydroxyl, or both. It is particularly preferred that R₁ represents methyl or ethyl, preferably methyl.

The compounds according to the present invention are antimuscarinic agents. “Antimuscarinic agents” refer to muscarinic receptor antagonists. Examples of known antimuscarinic agents include tolterodine, hydroxytolterodine, 2-(diisopropylamino) ethyl-1-phenylcyclopentanecarboxylate, propiverine, oxybutynin, trospium, temiverine, and ipratropium. Methods of assaying for muscarinic receptor activity are described, for example, by N. Watson et al in Eur. J. Pharmacol., 285(2), 135-142 (1995).

Propiverine is 1-methyl-4-piperidyl α,α-diphenyl- α-(n-propoxy)acetate and is disclosed in East German Patent 106,643 and in CAS 82-155841s (1975). Oxybutynin is 4-(diethylamino)-2-butynylalphaphenylcyclohexaneglycolate and is disclosed in UK Patent 940,540. Trospium is 3α-hydroxyspiro[1αH,SαH-nortropane30 8,1′pyrrolidinium]chloride benzilate and is disclosed in U.S. Pat. No. 3,480,623. Temiverine is 3S benzeneacetic acid, α-cyclohexyl-α-hydroxy-, 4-(diethylamino)-1,1-dimethyl-2-butynyl ester and is disclosed in U.S. Pat. No. 5,036,098. Ipratropium is 8-isopropylnoratropine methobromide and is disclosed in U.S. Pat. No. 3,505,337.

The compounds of formulae I-V have anti-cholinergic properties and unexpectedly exhibit prolonged activity in the gut relative to non-quarternized compounds. Thus, the compounds of formulae I-V are useful for the treatment of acetylcholine-mediated disorders.

The second pharmaceutical agent includes one or more compounds selected from Adenosine A₂ₐ Receptor Agonists, D2-Dopamine Receptor Agonists, Phosphodiesterase Inhibitors (PDE's), corticosteroids, norepinephrine reuptake inhibitors, 4-hydroxy-7-[2-[2-[3-[2-phenylethoxy]-propylsulphonyl]ethylamino]ethyl]-1,3-benzothiazol-2(3H)-one, and pharmaceutically acceptable salts thereof, and and non-quarternized antimuscarinic compounds.

Adenosine A_{2A} Receptor Agonists

The class of adenosine A_{2A} receptor agonists useful in the novel combinations of therapeutic agents of the present invention include compounds which exhibit an acceptably high affinity for the A_{2A}-subtype of adenosine receptor and acceptably high therapeutic index for lung effects compared with effects in the periphery after inhalation. Adenosine has a wide range of physiologic activities, including immune and inflammatory responses, which are receptor mediated and involve interaction with at least four types of plasma membrane receptors. These receptors are commonly referred to as A₁, A_{2A}, A_{2B}, and As. Adenosine and its analogs have been found to possess a broad spectrum of anti-inflammatory activity that involves a significant variety of immune and inflammatory cells, including neutrophils and eosinophils.

Examples of adenosine A_{2A} receptor agonists are described in U.S. Pat. Nos. 5,605,908, 5,998,404, 5,821,249, and 5,861,423, each of which are incorporated herein in their entirety; and International Applications WO 99/34804; WO 99/67263; WO 97/08146; WO 99/67263; WO 00/51970; WO02/095273; and WO02/096462, each of which are incorporated herein in their entirety.

Other descriptions of the adenosine A_{2A} receptor agonists may be found in Visser et al., “Apparent Involvement of the A_{2A} Subtype Adenosine Receptor in the Anti-inflammatory Interactions of CGS 2168O, Cyclopentyladenosine, and IB-MECA with Human Neutrophils,” Biochem. Pharmacol., 60 993-999, 2000.; Martin et al., “Pharmacology of 2-Cyclohexyl-methylidene-hydrazino adenosine (WRC-0470), a Novel, Short-Acting Adenosine A_{2A} Receptor Agonist That Produces Selective Coronary Vasodilation,” Drug Dev. Res.” 40 313-324, 1997; Keeling et al., “The Discovery and Synthesis of Highly Potent, A_{2A} Receptor Agonists,” Bioorg. Med. Chem. Lett. 10 403-406, 2000; and Kull et al., “Differences in the Order of Potency for Agonist But Not; Antagonists at Human and Rat Adenosine A_{2A} Receptors,” Biochem. Pharmacol. 57 65-75, 1999; Sullivan and Linden, “Role of A_{2A} Adenosine Receptors in Inflammation,” Drug.

Examples of specific adenosine A_{2A} receptor agonists include, but are not limited to, N9-[(2R,3R,45,5R)-2-{2-(aminomethyl)-6-[(2,2-diphenylethyl)amino]-9H-purin-9-yl}-5 (methoxymethyl)tetrahydro-3,4-furandiol; N-{[9-[(2R,3R,4S,5R)-3,4-dihydroxy-5-(methoxymethyl)tetrahydro-2-furanyl]-6-[(2,2 diphenylethyl) amino]-9H-purin-2-yl]methyl}-2-phenylacetamide; N-{[9-[(2R,3R,45,5R)-3,4-dihydroxy-5-(methoxymethyl)tetrahydro-2-furanyl]-6-[(2,2 10 diphenylethyl)amino]-9H-purin-2-yl]methyl}benzamide; N-t [9-[(2R,3R,4S,5R)-3,4-dihydroxy-5-(methoxymethyl)tetrahydro-2-furanyl]-6-[(2,2 diphenylethyl)amino]-9H-purin-2-yl]methyl}benzenesulfonamide; (2R,3R,4S, 5R)-2-[2-(benzylamino)methyl]-6-[(2,2-diphenylethyl)amino]-9H-purin-9-yl]5-(methoxymethyl)tetrahydro-3,4-furandiol; 1,5(2R,3R,45,5R)-2-[2-(cyclohexylamino)methyl]-6-[(2,2-diphenylethyl)amino]-9H-purin 9-yl]-5-(methoxymethyl)tetrahydro-3,4-furandiol; (2R,3R,45,5R)-2-[2-{[(cyclohexylmethyl)amino]methyl}-6-[(2,2-diphenylethyl)-amino]9H-purin-9-yl]-5-(methoxymethyl)tetrahydro-3,4-furandiol; (2R,3R,4S,5R)-2-[2-[(cyclopentylamino)methyl]-6-[(2,2-diphenylethyl)amino]-9H 20 purin-9-yl]-5-(methoxymethyl)tetrahydro-3,4-furandiol; N-{[9-[(2R,3R,4S,SR)-3,4-dihydroxy-5-(methoxymethyl)tetrahydro-2-furanyl]-6-[(2,2 diphenylethyl)amino]-9H-purin-2-yl]methyl}-1-propanesulfonamide; (2R,3R,4S,5R)-2-{6-L(2,2-diphenylethyl)amino]-2-[(isopropylamino)methyl]-9H-purin 9-yl}-5-(methoxymethyl)tetrahydro-3,4-furandiol; 25 (2R,3R,4S,SR)-2-{2-(2-aminoethyl)-6-[(2,2-diphenylethyl)amino]-2 [(isopropylamino)methyl]-9H-purin-9-yl}-5-(methoxymethyl)tetrahydro-3,4-furandiol; (2R,3R,4S,SR)-2-{2-[2-(cyclohexylamino)ethyl]-6-[(2,2-diphenylethyl)amino]-2 [(isopropylamino)methyl]-9H-purin-9-yl}-5-(methoxymethyl)tetrahydro-3,4-furandiol; N-(2-{9-[(2R,3R,4S,SR)-3,4-dihydroxy-5-(methoxymethyl)tetrahydro-2-furanyl]-6-[(2,2 30 diphenylethyl)amino]-9H-purin-2-yl}methyl)benzenesulfonamide; (2R,3R,45,5R)-2-(6-[(2,2-diphenylethyl)amino]-2-[2-(isopropylamino)ethyl]-9H-purin 9-yl}-5-(methoxymethyl)tetrahydro-3,4-furandiol; N-({9-[(2R, 3R,4S,5R)-3,4-dihydroxy-5-(methoxymethyl)tetrahydro-2-furanyl]-6-[(2,2 diphenylethyl)amino]-9H-purin-2-yl}methyl)-2-methyl-1-propanesulfonamide; 9-[(2R,3R,45,5R)-3,4-dihydroxy-5-(hydroxymethyl)-tetrahydro-2-furanyl]-6-[(2,2 diphenylethyl)amino]-N-[2-(1-piperdinyl)ethyl]-9H-purine-2-carboxamide; 9-[(2R,3R,45,5R)-3,4-dihydroxy-5-(hydroxymethyl)-tetrahydro-2-furanyl]-6-[(2,2 diphenylethyl)amino]-N-phenylethyl-9H-purine-2-carboxamide; 9-[(2R,3R,45,5R)-3,4-dihydroxy-5-(hydroxymethyl)-tetrahydro-2-furanyl]-6-[(2,2 10 diphenylethyl)amino]-N-[2-(4-isopropyl-1-piperdinyl)ethyl]-9H-purine-2-carboxamide; 9-[(2R,3R,45,5R)-3,4-dihydroxy-5-(hydroxymethyl)-tetrahydro-2-furanyl]-6-[(2,2 diphenylethyl)amino]-N-[3-(1-pyrrolidinyl)propyl]-9H-purine-2-carboxamide; 9-[(2R,3R,45,5R)-3,4-dihydroxy-5-(hydroxymethyl)-tetrahydro-2-furanyl]-6-[(2,2 diphenylethyl)amino]-N-[2-(4-morpholinyl)ethyl]-9H-purine-2-carboxamide; 15 9-[(2R,3R, 45,5R)-3,4-dihydroxy-5-(hydroxymethyl)-tetrahydro-2-furanyl]-6-[(2,2 diphenylethyl)amino]-N-(2-pyridinylmethyl]-9H-purine-2-carboxamide; 9-[(2R,3R,45,5R)-3,4-dihydroxy-5-(hydroxymethyl)-tetrahydro-2-furanyl]-6-[(2,2 diphenylethyl)amino]-N-[2-(2-pyridinyl)ethyl]-9H-purine-2-carboxamide; 9-[(2R,3R,45,5R)-3,4-dihydroxy-5-(hydroxymethyl)-tetrahydro-2-furanyl]-N-[2 20 (dimethylamino)ethyl]-6-[(2,2-diphenylethyl)amino]-9H-purine-2-carboxamide; N-({9-[(2R,3R,45,5R)-3,4-dihydroxy-5-(hydroxymethyl)tetrahydro-2-furanyl]-6-[(2,2 diphenylethyl)amino]-9H-purin-2-yl}methyl)-2-methyl-1-propanesulfonamide; N-([9-[(2R,3R,4S,5R)-3,4-dihydroxy-5-(hydroxymethyl)tetrahydro-2-furanyl]-6 (phenylethylamino)-9H-purin-2-yl]methyl}benzenesulfonamide; 25 N-((9-[(2R,3R,45,5R)-3,4-dihydroxy-5-(hydroxylmethyl)tetrahydro-2-furanyl]-6-[(1 naphthylmethyl)amino]-9H-purin-2-yl}methyl)benzenesulfonamide; 2-[cyclopentyl(isopropyl)amino]-N-((9-[(2R,3R,4S,5R)-3,4-dihydroxy-5-(hydroxy methyl)tetrahydro-2-furanyl]-6-[(2,2-diphenylethyl)amino]-9H-purin-2-yl methyl)ethanesulfonamide; 30 (2S,35,4R,5R)-5-(2-([(benzylsulfonyl)amino]methyl}-6-[(2,2-diphenylethyl)-amino]9H-purin-9-yl}-N-ethyl-3,4-dihydroxytetrahydro-2-furancarboxamide; (2S,3S,4R,5R)-5-(6-[(2,2-diphenylethyl)amino]-2-{[(propylsulfonyl)amino]-methyl}-9H purin-9-yl 3-N-ethyl-3,4-dihydroxytetrahydro-2-furancarboxamide; (2S,3S,4R,5R)-5-(6-[(2,2-diphenylethyl)amino]-2-{[(isopropylsulfonyl)amino]-methyl}9H-purin-9-yl}-N-ethyl-3,4-dihydroxytetrahydro-2-furancarboxamide; 5 (2S,3 S,4R,5R)-5-(6-[(2,2-diphenylethyl)amino]-2-{[(phenylsulfonyl)amino]-methyl}9H-purin-9-yl}-N-ethyl-3,4-dihydroxytetrahydro-2-furancarboxamide; (2S,3S,4R,5R)-5-{2-{[([1,1′-biphenyl]-4-ylsulfonyl)amino]methyl}-6-[(2,2 diphenylethyl amino]-9H-purin-9-yl}-N-ethyl-3,4-dihydroxytetrahydro-2-furancarboxamide; (2S,3S,4R,5R)-5-(6-[(2,2-diphenylethyl)amino]-2-{[(naphthylsulfonyl)amino]-methyl}10 9H-purin-9-yl)-N-ethyl-3,4-dihydroxytetrahydro-2-furancarboxamide; N-({9-[(2R,3R,45,5R)-3,4-dihydroxy-5-(hydroxymethyl)tetrahydro-2-furanyl]-6-[(2,2 diphenylethyl)amino]-9H-purin-2-yl}methyl)-N-[2-di-isopropylamino)ethyl]urea; N-({9-[(2R,3R,4S,5R)-3,4-ditydroxy-5-(hydroxymethyl)tetrahydro-2-furanyl]-6-[(2,2 diphenylethyl)amino]-9H-purin-2-yl}methyl)-N-[2-(1-piperidinyl)ethyl]urea; 15 (2S,3S,4R,5R)-5-{2-{[({[2-(di-isopropylamino)ethyl]amino}carbonyl)amino]-methyl}-6 [(2,2-diphenylethyl)amino]-9H-purin-9-yl}-N-ethyl-3,4-dihydroxytetrahydro-2 furancarboxamide; (2S,3S,4R,5R)-5-(6-[(2,2-diphenylethyl)amino]-{2-{[({[2-(1-piperidinyl)ethyl]-amino}carbonyl)amino]methyl}-9H-purin-9-yl}-N-ethyl-3,4-dihydroxytetrahydro-2 furancarboxamide; N-({6-{[2,2-bis(4-chlorophenyl)ethyl]amino}-9-[(2R,3R,4S,5R)-3,4-dihydroxy-5 (hydroxymethyl)tetrahydro-2-furanyl]-6-[(2,2-diphenylethyl)amino]-9H-purin-2-yl}methyl) N-[2-(2-di-isopropylamino)ethyl]urea; N-[2-(dicyclobutylamino)ethyl]-N-({9-[(2R,3R,45,5R)-3,4-dihydroxy-5 25 (hydroxymethyl)tetrahydro-2-furanyl]-6-[(2,2-diphenylethyl)amino]-9H-purin-2 yl}methyl)urea; 6-(2,2-diphenylethyl)amino]-9-{(2R, 3R,45,55)-5-[(ethylamino)carbonyl]-3,4dihydroxytetrahydro-2-furanyl}-N-[2-(1-piperidinyl)ethyl]-9H-purine-2-carboxamide; 6-[(2,2-diphenylethyl)amino]-9-{(2R,3R,4S,SS)-5-[(ethylamino)carbonyl]-3,4 30 dihydroxytetrahydro-2-furanyl]-N-[2-(4-isopropyl-1-piperidinyl)ethyl]-9H-purine-2 carboxamide; 6-[(2,2-diphenylethyl)amino]-9-{(2R,3R,4S,5 S)-5-[(ethylamino)carbonyl]-3,4 dihydroxytetrahydro-2-furanyl}-N-{2-[({[2-(1 piperidinyl)ethyl]amino}carbonyl)amino]ethyl}-9H-purine-2-carboxy amide; N-{2-[({[2-(di-isopropylamino)ethyl]amino}carbonyl)amino]ethyl}-6-[(2,2 5 diphenylethyl)amino]-9-{(2R,3R,45,5S)-5-[(ethylamino)carbonyl]-3,4-dihydroxytetrahydro-2 furanyl}-9H-purine-2-carboxamide; 9-[(2R,3R,4S,5R)-3,4-dihydroxy-5-(hydroxymethyl)tetrahydro-2-furanyl]-6-[(2,2 diphenylethyl)amino]-N{2-[({[2-(1-piperidinyl)ethyl]amino}carbonyl)amino]ethyl}-9H purine-2-carboxamide; 10 9-[(2R,3R,4S,5R)-3,4-dihydroxy-5-(hydroxymethyl)tetrahydro-2-furanyl]-N-{2-[({[2 (di-isopropylamino)ethyl]amino}carbonyl)amino]ethyl}-6-[(2,2-diphenylethyl)amino]-9H purine-2-carboxamide; 6-[(2,2-diphenylethyl)amino]-9-{(2R, 3R,4S,5S)-5-[(ethylamino)carbonyl]-3,4dihydroxytetrahydro-2-furanyl}-N-{2-[([2-(4-isopropyl-1-piperidinyl)ethyl]amino}15 carbonyl)-amino]ethyl}-9H-purine-2-carboxamide; N-(2-{[({2-[cyclopentyl(isopropyl)amino]ethyl}amino)carbonyl]amino}ethyl)-6-[(2,2diphenylethyl)amino]-9-{(2R,3R,4S,55)-5-[(ethylamino)carbonyl]-3,4-dihydroxytetra-hydro-2 furanyl}-9H-purine-2-carboxamide; and N-(2-{[({2-[cyclohexyl(isopropyl)amino]ethyl}amino)carbonyl]amino} ethyl)-6-[(2,2diphenylethyl)amino]-9-{(2R,3R,4S,5S)-5-[(ethylamino)carbonyl]-3,4-dihydroxytetra-hydro-2 furanyl}-9H-purine-2-carboxamide, and the pharmaceutically acceptable salts and solvates thereof.

D2-Dopamine Receptor Agonists

The class of dopamine D2-receptor agonists useful in the novel combinations of therapeutic agents of the present invention include compounds which exhibit an acceptably high affinity for the D2 subtype of dopamine receptor. There are two isoforms of the D2 subtype, often referred to as D2 long and D2 short, based on diflerences in length of their third cytoplasmic loops. Dopamine D2-receptors couple to multiple effector systems, including the inhibition of adenylyl cyclase activity. It is believed that activation of dopamine receptors of this class leads to suppression of the activity of sensory afferent nerves in the airway, which in turn reduces the consequences of afferent nerve activity in this context, namely, reduction of dyspnea and of reflex events for example suppression of the release of the neurotransmitter acetylcholine and of other transmitters, which mediate efferent nerve activity in the lung.

Examples of Dopamine D2-receptor agonists may be found in WO 99/136095; U.S. Pat. Nos. 4,622,398, 5,235,055, 5,382,596, 5,633,376, 5,674,909, 5,733,908, 5,747,513, 6,080,768, 5,750,556, 5,814,628, and 5,972,958, each of which in incorporated herein in their entirety and in International Applications WO 95/33729, WO 96/04910, WO 97/136893, WO 98/08817, WO 98/08843, WO 98/35945, WO 98/35948, WO 98/38155, WO 99/157119, WO 00/16777, WO02/096422, EP 409,048, EP 875,512, and EP 899,267, each of which in incorporated herein in their entirety.

Specific examples of the dopamine D2-receptor agonists include, but are not limited to, alentemol; apomorphine; biperiden; bromocriptine; cabergoline; carmoxirole; ciiadopa; dopexamine; fenoldopam; ibopamine; levodopa; lisuride; 5 methylenedioxypropyinoraporphine; naxagolide; N-allyinoraporphine; pergolide; pramipexole; propylnorapomorphine; protokylol; quinagolide; quinpirole; ropinirole; roxindole; talipexole; terguride; trihexyphenidyl; trihydroxyaporphine; and pharmaceutically acceptable salts thereof.

Some specific examples of pharmaceutically acceptable salts of the dopamine D2-receptor agonists include, but are not limited to, alentemol hydrobromide; apomorphine hydrochloride; N-methylapomorphinium bromide; biperiden hydrochloride; biperiden lactate; bromocriptine mesylate; cabergoline diphosphate; carmoxirole hydrochloride; ciladopa hydrochloride; dopexamine dihydrochloride; dopexamine dihydrobromide; fenoldopam hydrobromide; fenoldopam mesylate; ibopamine hydrochloride; lisuride maleate; naxagolide hydrochloride; 15 pergolide mesylate; pramipexole dihydrochloride; protokylol hydrochloride; quinagolide hydrochloride; quinpirole hydrochloride; ropinirole hydrochloride; roxindole hydrochloride; roxindole mesylate; talipexole dihydrochloride; terguride hydrogen maleate; terguride hydrogen maleate hydrate; and trihexyphenidyl hydrochloride.

PDE Inhibitors

PDE is involved in numerous functional pathways in tissues throughout the body. Agents such as theophylline and caffeine have been recognized as non-specific PDE inhibitors for several decades. See GOODMAN & GILMAN'S THE PHARMACOLOGICAL BASIS OF THERAPEUTICS, 832-4, (Joel G. Hardman et al. eds., 9^{th} ed. 1996). More recently, classes of PDE inhibitors exhibiting more or less specificity for one or more of the mutiple isoforms of PDE have been described, and produce function-specific effects. For example, PDE-III specific inhibitors produce vascular and airway dilation, inhibition of platelet aggregation, stimulation of lipolysis, and inhibition of cytokine production. Id. PDE-IV specific inhibitors produce airway smooth muscle relaxation, inhibition of inflammatory mediator release, CNS modulation, and gastric acid secretion.

The PDE inhibitor or pharmaceutically acceptable salt is any PDE inhibitor including isozyme-selective inhibitors of PDE-I, PDE-II, PDE-III, PDE-IV, PDE-V, PDE-VI and PDE-VII, and also PDE-III/IV dual inhibitors. The terms “phosphodiestrease inhibitor” and “PDE inhibitor” as used interchangeably herein denote a compound that reduces the physiological effect of a phosphodisterase enzyme, thus slowing the degradation of cyclic AMP (cAMP) and cyclic (cGMP). Such an inhibitor may be specific (i.e. selective) for a particular isozyme of phosphodiesterase, or may be substantially non-specific (i.e. non-selective), that is, effective to a large extent on two or more isoforms of phosphodiesterase.

The term “PDE-I inhibitor” denotes a compound that is capable of reducing the physiological effect of the PDE-I isoform of phosphodiesterase preferentially over other isoforms of phosphodiesterase.

The term “PDE-II inhibitor” denotes a compound that is capable of reducing the physiological effect of the PDE-II isoform of phosphodiesterase preferentially over other isoforms of phosphodiesterase.

The term “PDE-III inhibitor” denotes a compound that is capable of reducing the physiological effect of the PDE-III isoform of phosphodiesterase preferentially over other isoforms of phosphodiesterase.

The term “PDE-IV inhibitor” denotes a compound that is capable of reducing the physiological effect of the PDE-IV isoform of phosphodiesterase preferentially over other isoforms of phosphodiesterase.

The term “PDE-V inhibitor” denotes a compound that is capable of reducing the physiological effect of the PDE-V isoform of phosphodiesterase preferentially over other isoforms of phosphodiesterase.

The term “PDE-VI inhibitor” denotes a compound that is capable of reducing the physiological effect of the PDE-VI isoform of phosphodiesterase preferentially over other isoforms of phosphodiesterase.

The term “PDE-VII inhibitor” denotes a compound that is capable of reducing the physiological effect of the PDE-VII isoform of phosphodiesterase preferentially over other isoforms of phosphodiesterase.

The term “PDE-III/IV dual inhibitor” denotes a compound that is capable of reducing the physiological effect of the PDE-III and PDE-IV isoforms of phosphodiesterase preferentially over other isoforms of phosphodiesterase.

Examples of PDE inhibitors are described in U.S. Pat. Nos. 6,333,354, and 6,331,543, each of which is incorporated herein in their entirety.

Specific examples of PDE inhibitors include, but are not limited to, non-specific PDE inhibitors such as Theophylline, Dipyridamole, TRENTAL (pentoxifylline), Hoechst Marion Roussel, (Bad Soden, Germany); and Isobutyl methylxanthine (IBMX);

PDE-I inhibitors such as VINPOCETINE, KS-505a, W-7, and Phenothiazines;

PDE-II inhibitors such as EHNA; PDE-IV inhibitors such as RO-20-1724, DENBUFYLLINE, OXAGRELATE, NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840 and the S-enantiomer thereof, CT1731, SKF-107806, PICLAMILAST, RS-17597, RS-25344-000, SB-207499, TIBENELAST, SB-210667, SB-211572, SB-211600, SB-212066, SB-212179 and GW-3600, in particular MOPIDAMOL, ANAGRELIDE, IBUDILAST, AMRINONE, PIMOBENDAN, CILOSTAZOL, LAS-31025-Almirall; Propentophylline (PPF also known as HWA-285); L-826,141; QUAZINONE and N-(3,5-dichloropyrid-4-yl)-3-cyclopropylmethoxy-4-difluoromethoxybenzamide; and CILOMILAST (Ariflo®, SB 207499) c-4-cyano-4-(3-cyclopentyloxy-4-methoxyphenyl-r-1-cyclohexane carboxylic acid), SmithKline Beecham Pharmaceuticals plc, (Harlow, UK), having the structure:

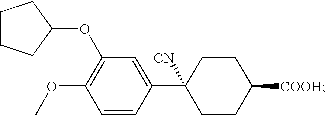

D4418; D4396; SCH351591; MESOPRAM, Chiroscience and Schering-Plough; ROLIPRAM [4-(3-cyclopentenyloxy-4-methoxyphenyl)-2-pyrrolidone], CAS [61413-54-5], Schering AG (Berlin, Germany), having the structure:

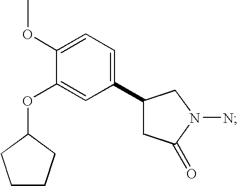

YM976 (4-(3-chlorophenyl)-1,7-diethylpyrido[2,3-d]pyrimidin-2(1H)-one Yamanouchi Pharmaceutical Co. Ltd. (Tsukuba, Japan) having the structure:

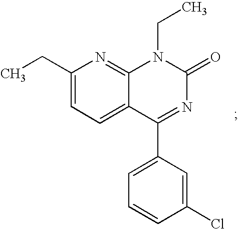

RP73401 (3-cyclopentyloxy-N-(3,5-dichloro-4-pyridyl)-4-methoxybenzamide);

CT-2450, ((R)-N-{4-[1-(3-cyclopentyloxy-4-methoxyphenyl)-2-(4-pyridyl)ethyl]phenyl}N′-ethylurea), Celltech Group plc (Berkshire, GB), having the structure:

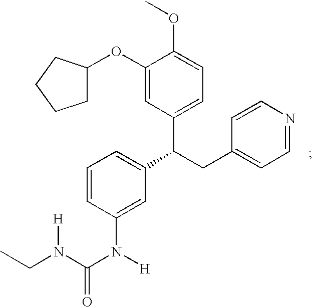

CT-3405, Celltech Group plc (Berkshire, GB), having the structure:

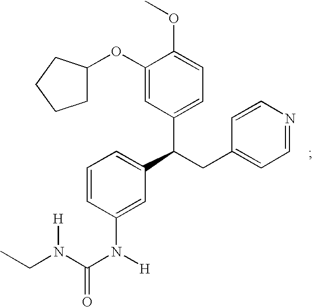

and compounds described in U.S. Pat. No. 5,712,298, Amschler, BYK Gulden Lomberg Chemische Fabrik GmbH (Konstanz, Germany), particularly the compound ROFLUMILAST (RP 73401), (benzamide 3-(cyclopropylmethoxy)-N-(3,5-dichloro-4-(difluoromethoxy)-(9Cl)), having the structure:

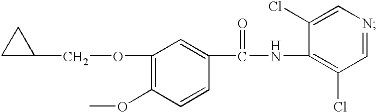

and BENAFENTRINE (6-(p-acetamidophenyl)-1,2,3,4,4a,10b-hexahydro-8,9-dimethoxy-2-methyl-benzo[c][1,6]naphthyridine); BAY 19-8004, Bayer; Pumafentrine; INS-365; AWD 12-281, Asta Medica (now known as Elbion); compounds described in U.S. Pat. No. 6,384,236, Pfizer; CDC-801 and CDC-998, Celgene; and 5CC (catechole hydrazine type derivatives), Cheil Je Dang Corp; PDE-III/IV dual inhibitors such as TREQUINSINE, ORG-30029, L-686398, SDZ-ISQ-844, ORG-20241, EMD-54622; ZARDAVERINE; TOLAFENTRINE, Byk Gulden Pharmaceuticals (Konstanz, Germany);

PDE-III inhibitors such as AMRINONE, SULMAZOLE, AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, in particular ENOXIMONE and MILRINONE; VESNARINONE; INDOLIDANE; QUAZINONE; MOTAPIZONE; SK&F 94836; MKS 492; CI-930 (4,5-dihydro-6-[4-(1H-imidazol-1-yl)-phenyl]-5-methyl-3(2H)-pyridazinone), Tanabe Seiyaku (Osaka, Japan); and

ATZ-1993 having the structure:

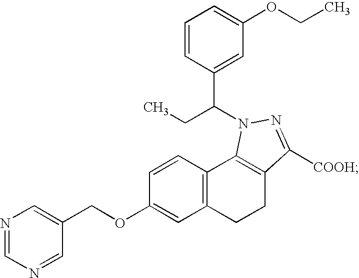

OLPRINONE (E-1020:1,2-Dihydro-6-methyl-2-oxo-5-[imidazo(1,2-a)pyridin-6-yl]-3-pyridine carbonitrile hydrochloride monohydrate); and CILOSTAZO;

PDE IV Inhibitors are described in WO 02/096423 and WO 02/096463, each of which are incorporated herein in their entirety. Specific PDE IV inhibitors include, but are not limited to, 9-cyclopentyl-5,6-dihydro-7-ethyl-3-phenyl-9H-pyrazolo[3,4-c1-1,2,4-triazolo[4,3 20 alpyridine; 9-cyclopenyl-5,6-dihydro-7-ethyl-3-(furan-2-yl)-9H-pyrazolo[3,4-c]-1,2,4-triazolo[4,3-a]pyridine; 9-cyclopentyl-5,6-dihydro-7-ethyl-3-(2-pyridyl)-9H-pyrazolo[3,4-c]-1,2,4-triazolo[4,3-3pyridine; 9-cyclopentyl-5,6-dthydro-7-ethyl-3-(4-pyridyl)-9H-pyrazolo[3,4-c]-1,2,4-triazolo[4,3 or]pyridine; 9-cyclopentyl-5,6-dihydro-7-ethyl-3-(3-thienyl)-9H-pyrazolo[3,4-c]-1,2,4-triazolo[4,3alpyridine; 3-benzyl-9-cyclopentyl-5,6-dihydro-7-ethyl-9H-pyrazolo[3,4-c]-1,2,4-triazolo[4,3]pyridine; 9-cyclopentyl-5,6-dihydro-7-ethyl-3-propyl-9H-pyrazolo[3,4-c]-1,2,4-triazolo[4,3-]pyridine; 3,9-dicyclopentyl-5,6-dibydro-7-ethyl-9H-pyrazolo[3,4-c]-1,2,4-triazolo[4,3-]pyridine; 9-cyclopentyl-5,6-dihydro-7-ethyl-3-(1-methylcyclohex-1-yl)-9H-pyrazolo[3,4-c]-1,2,4-triazolo[4,3-]pyridine; 3-(teff-butyl)-9-cyclopentyl-5,6-dthydro-7-ethyl-9H-pyrazolo[3,4-c]-1,2,4-triazolo[4,3-]pyridine; 9-cyclopentyl-5,6-dihydro-7-ethyl-3-(2-methylphenyl)-9H-pyrazolo[3,4-c]-1,2,4-triazolo[4,3-]pyridine; 109-cyclopentyl-5,6-dthydro-7-ethyl-3-(2-methoxyphenyl)-9H-pyrazolo[3,4-c]-1,2,4 triazolo[4,3-]pyridine; 9-cyclopentyl-5,6-dthydro-7-ethyl-3-(thien-2-yl)-9H-pyrazolo[3,4-c]1,2,4-triazolo[4,3-]pyridine; 3-2-chlorophenyl)-9-cyclopentyi-5,6-dlhydro-7-ethyl-9H-pyrazolo[3,4-c]-1,2,4-triazolo[4,3-]pyridine; 9-cyclopentyl-5,6-dihydro-7-ethyl-3-(2-iodophenyl)-9H-pyrazolo[3,4-c]-1,2,4-triazolo[4,3-]pyridine; 9-cyclopentyl-5,6-dihydro-7-ethyl-3-(2-trifluoromethylphenyl)-9H-pyrazolo[3,4-c]-1,2,4-triazolo[4,3-]pyridine; and 20 5,6-dihydro-7-ethyl-9-(4-fluorophenyl)-3-(1-methylcyclohex-1-yl)-9H-pyrazolo[3,4-c]1,2,4-triazolol4,3-]pyridine;

PDE V Inhibitors such as dipyridamole, MY-5445, RX-RA-69, SCH-51866, KT-734, VESNARINONE, ZAPRINAST, SKF-96231, ER-21355, BF/GP-385, NM-702 and SILDENAFI; and

PDE VI Inhibitors such as dipyridamole and zaprinast.

Corticosteroids

Examples of Corticosteroids include anti-inflammatory Corticosteroids. Examples of anti-inflammatory Corticosteroids are described in WO 92/13872, GB-2088877, DE 2,323,215, EP 57,401, and U.S. Pat. Nos. 3,929,768, 4,472,393, each of which are incorporated herein in their entirety. Specific examples of anti-inflammatory Corticosteroids include, but are not limited to, rofleponide, fluticasone propionate, budesonide, and mometasome.

Norepinephrine Reuptake Inhibitors

Examples of norepinephrine reuptake inhibitors (both selective and not selective) include, but are not limited to the following: tandamine (CAS 42408-80-0; U.S. Pat. No. 3,904,617-, U.S. Pat. No. 4,118,394), pirandamine (CAS 42408-79-7; U.S. Pat. No. 3,995,052), ciclazindol (CAS 37751-39-6; U.S. Pat. No. 3,891,644; U.S. Pat. No. 3,957,819; U.S. Pat. No. 3,976,645), fluparoxan (U.S. Pat. No. 4,880,801), lortalamine (CAS 70384-91-7; U.S. Pat. No. 4,201,783), talsupram (CAS 21489-20 3), talopram (CAS 7182-51-6), prindamine, nomifensine (U.S. Pat. No. 3,577,424), viloxazine (U.S. Pat. No. 3,712,890), tomoxetine (U.S. Pat. No. 4,314,081), duloxetine (U.S. Pat. No. 5,023,269), venlafaxine (U.S. Pat. No. 4,535,186), milnacipran (U.S. Pat. No. 4,478,836), reboxetine (U.S. Pat. No. 4,229,449), and Milnacipran.

Non-Quarternized Antimuscarinic Agents

Examples of antimuscarinic agents include, but are not limited to the following: tolterodine, propiverine, oxybutynin, trospium, darifenacin, temiverine, ipratropium.

The second pharmaceutical agent may also include 4-Hydroxy-7-[2-[2-[3-[2-phenylethoxy]propylsulphonyl]ethylamino]ethyl]-1,3-benzothiazol-2(3H)-one and pharmaceutically acceptable salts thereof which are described in WO 93/24473.

The combination therapy of the present invention is useful in treating mammals, including man. The compounds according to the invention, in the form of free base or salts with pharmaceutically acceptable acids, or solutions thereof, can be brought into suitable dosage forms, such as compositions for administration through the oral, rectal, transdermal, parenteral, nasal, or pulmonary route in accordance with accepted pharmaceutical procedures. In particular, the compositions of the combination therapy may be administered via inhalation or insufflaion.

Such pharmaceutical compositions according to the invention may include the first and second pharmaceutical agents according to the invention in association with compatible pharmaceutically acceptable carrier materials, or diluents, as is well known in the art. The carriers may be any inert material, organic or inorganic, suitable for administration, such as: water, gelatin, gum arabicum, lactose, microcrystalline cellulose, starch, sodium starch glycolate, calcium hydrogen phosphate, magnesium stearate, talcum, colloidal silicon dioxide, and the like. Such compositions may also contain other pharmaceutically active agents, and conventional additives such as stabilizers, wetting agents, emulsifiers, flavoring agents, buffers, binders, disintegrants, lubricants, glidants, antiadherents, propellants, and the like. The carrier, e.g., non-active ingredient, can be just (sterile) water with the pH adjusted to where the active pharmaceutical agent is very soluble. It is preferred that the pH be at or near 7. Alternatively and preferably, the non-active carrier agent should be physiological saline with the pH adjusted appropriately.

The first and second pharmaceutical agents according to the present invention can be administered in any suitable way. The compounds according to the invention can be made up in solid or liquid form, such as tablets, capsules, powders, syrups, elixirs and the like, aerosols, sterile solutions, suspensions or emulsions, and the like. The compounds are advantageously administered via inhalation or insufflation. When the administration form is inhalation or insufflation, the compounds are preferably in the form of either an aerosol or a powder.

The term “effective amount” refers to a therapeutically effective amount for treating asthma, chronic obstructive pulmonary disease (COPD), allergic rhinitis, or infectious rhinitis. The terms “therapy” and “therapeutically” encompass all kinds of treatments, including prophylaxis. In particular, “therapeutically effective” means that it is effective in preventing or arresting COPD. Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

For purposes of illustration, dosages are expressed for based on the inhalation of an aerosol solution, such as the product Atrovent Inhalation Aerosol (Boehringer Ingelheim). Adjustments in dosages for administration by other modes of inhaled administration are well known to those skilled in the art.

In general, a therapeutically effective amount of the first and/or second pharmaceutical agent (e.g., combined or separate) is from about 1 μg to about 1,000 μg, e.g., from about 10 μg to about 1,000 μg or from about 100 μg to about 1000 μg. However, the exact dosage of the specific compound according to the invention will vary depending on the combination of the first and second pharmaceutical agents, its potency, the mode of administration, the age and weight of the patient and the severity of the condition to be treated. The daily dosage may, for example, range from about 0.01 μg to about 10 μg per kg of body weight, administered singly or multiply in doses e.g. from about 1 μg to about 1,000 μg each. The combination therapy can be administered from one to four times daily, e.g., once or twice daily.

The dosage form for inhalation can be an aerosol. The minimum amount of an aerosol delivery is about 0.2 ml and the maximum aerosol delivery is about 5 ml. The concentration of the compounds according to the invention may vary as long as the total amount of spray delivered is within the about 0.2 to about 5 ml amount and it delivers a therapeutically effective amount of the desired compounds. It is well known to those skilled in the art that if the concentration is higher, one gives a smaller dose to deliver the same effective amount.

The dosage form for inhalation can also be via intranasal spray. The minimum amount of an aerosol delivery is about 0.02 ml per nostril and the maximum aerosol delivery is about 0.2 ml per nostril. The concentration of the compounds according to the invention may vary as long as the total amount of spray delivered is within about 0.02 ml per nostril to about 0.2 ml per nostril, e.g., between about 0.05 ml per nostril and about 0.08 ml per nostril, and it delivers a therapeutically effective amount of the desired compounds.

Of course, the volume of aerosol or intranasal spray for delivering a therapeutically effective amount of the compound of formula I depends upon the concentration of the compound in the aerosol or intranasal spray, e.g., higher concentrations of the compound require smaller dosage volumes to deliver a therapeutically effective amount and lower concentrations of the compound require larger dosage volumes to deliver the same therapeutically effective amount.

Aerosols for inhalation of various pharmaceutical agents are well known to those skilled in the art, including many aerosols for treating asthma. Aerosols may be produced with a nebulizer. Typically, the nebulizer is charged with a carrier solution and the compound in an amount sufficient to effectively deliver a therapeutically effective amount of the antimuscarininc compound. For instance, depending upon the nebulizer and its operating conditions, the nebulizer may be charged with several hundred mg of antimuscarinic compound in order to deliver about 1 μg to about 1000 μg, e.g., from about 10 μg to about 1000 μg or from about 50 μg to about 500 μg, of the compound.

The dosage form for inhalation may also be in powder form. Powders for inhalation of various pharmaceutical agents are well known to those skilled in the art, including many powders for treating asthma. When the dosage form is a powder, the compounds according to the invention can be administered in pure form or diluted with an inert carrier. When an inert carrier is used, the compounds according to the invention are compounded such that the total amount of powder delivered delivers an “effective amount” of the compounds according to the invention. The actual concentration of the active compound may vary. If the concentration is lower, then more powder must be delivered; if the concentration is higher, less total material must be delivered to provide an effective amount of the active compound according to the invention.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

Although the combination therapy is described above as being administered via inhalation or insufflation, the compounds according to the present invention can be administered in any suitable way. The compounds according to the invention can be made up in solid or liquid form, such as tablets, capsules, powders, syrups, elixirs and the like, aerosols, sterile solutions, suspensions or emulsions, and the like.

Formulations for oral administration may be in the form of aqueous solutions and suspensions, in addition to solid tablet and capsule formulations. The aqueous solutions and suspensions may be prepared from sterile powders or granules. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants are well and widely known in the pharmaceutical art.

Pharmaceutical compositions of the first and second pharmaceutical agents, either individually or in combination, may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutical acceptable materials. The components pharmaceutical composition can be encapsulated or tableted for convenient administration.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Additionally, the first and second pharmaceutical agents may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

The first and second pharmaceutical agents may also be delivered by controlled-release formulation as may be provided in a dispersion of active compound in hydroxypropyl-methyl cellulose, or other methods known to those skilled in the art.

In the combination therapy, the first pharmaceutical agent, e.g., a compound of formulae I-V, or mixtures thereof, and the second pharmaceutical agent can be administered simultaneously or at separate intervals. When administered simultaneously the first and second pharmaceutical agents can be incorporated into a single pharmaceutical composition or into separate compositions, e.g., the first pharmaceutical agent, in one composition and the second pharmaceutical agent in another composition. Each of these compositions may be formulated with common excipients, diluents or carriers, and compressed into tablets, or formulated elixirs or solutions; and as sustained relief dosage forms and the like. The first and second pharmaceutical agents may be administered via different routes. For example, the first pharmaceutical agent may be administered via inhalation and the second pharmaceutical agent may be administered orally via tablet.

When separately administered, therapeutically effective amounts of the first and second pharmaceutical agents are administered on a different schedule. One may be administered before the other as long as the time between the two administrations falls within a therapeutically effective interval. A therapeutically effective interval is a period of time beginning when one of either (a) the first pharmaceutical agent or (b) the second pharmaceutical agent is administered to a mammal and ending at the limit of the beneficial effect in the treatment of the combination of (a) and (b).

Without further elaboration, it is believed that one skilled in the art can, using the preceding description, practice the present invention to its fullest extent. The foregoing detailed description is given for clearness of understanding only, and no unnecessary limitations should be understood therefrom, as modifications within the scope of the invention may become apparent to those skilled in the art.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

All temperatures are in degrees Celsius. Ether refers to diethyl ether. Physiological saline refers to a 0.9% aqueous 5 sodium chloride solution. When solvent pairs are used, the ratios of solvents used are volume/volume (v/v). When the solubility of a solid in a solvent is used the ratio of the solid to the solvent is weight/volume (wt/v).

### Definitions

All temperatures are in degrees Celsius.

Ether refers to diethyl ether.

Tolterodine refers to 2-[(1R)-3-(diisopropylamino)-1-phenylpropyl]-4-methylphenol also known as N,N-diisopropyl-3-(2-hydroxy-5-methylphenyl)-3-phenylpropylamine, a compound of the formula:

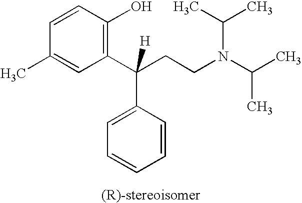

Hydroxytolterodine refers to 2-[(1R)-3-(diisopropylamino)-1-phenylpropyl]-4-(hydroxymethyl)phenol, a compound of the formula:

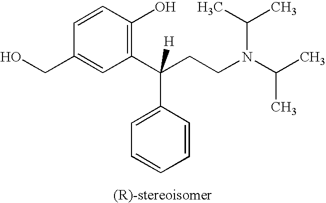

Physiological saline refers to a 0.9% aqueous sodium chloride solution.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

FEV₁ refers to Force Expiratory Volume in one second.

FEV₁/FVC refers to the ratio of the Force Expiratory Volume/Force Vital Capacity.

### Example 1

### Tolterodine Free Base

Tolterodine tartrate (2.1 g) is mixed with water (45 mL water) and toluene (2.5 mL). Sodium carbonate (800 mg) is added to the slurry. Sodium hydroxide (2.0 N, 1.5 mL) is added. The mixture is extracted three times with toluene (3 mL) saving the organic phase. Potassium carbonate is added to the organic phase to give the title compound in solution.

### Example 2

### (3R)-3-(2-Hydroxy-5-methylphenyl)-N,N-diisopropyl-N-methyl-3-phenylpropan-1-aminium iodide

Tolterodine free base (EXAMPLE 1, 0.5 M, 2.5 ml) is reacted with methyl iodide (1 ml). Acetonitrile (5 mL) is added to the mixture and stirred over night at 20-25°. The solvent is removed by blowing dry nitrogen. Acetone (1 mL) and hexane (2 ml) are added and the mixture filtered at 20-25° to give the title compound.

### Example 3

### (3R)-3-(2-Hydroxy-5-methylphenyl)-N,N-diisopropyl-N-methyl-3-phenylpropan-1-aminium bromide

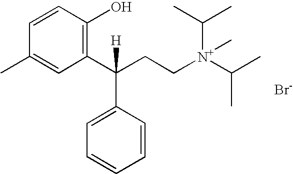

A sealed mixture of methyl bromide (100 g) and 2-[(1R)-3-(diisopropylamino)-1-phenylpropyl]-4-methylphenol (14 g) in acetone (100 mL) is stirred at 20-25° for 4 days. The mixture is cooled to −10° C. and the precipitate is filtered off and washed with ether and dried to give the title compound, mp 185°.

### Example 4

### (3R)-N-Ethyl-3-(2-hydroxy-5-methylphenyl)-N,N-diisopropyl-3-phenylpropan-1-aminium iodide

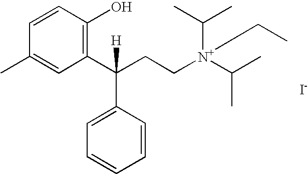

Following the general procedure of EXAMPLE 2 and making non critical variations, but starting with ethyl iodide the title compound is obtained.

### Example 5

### (3R)-3-(2-Hydroxy-5-methylphenyl)-N,N-diisopropyl-3-phenyl-N-propylpropan-1-aminium iodide

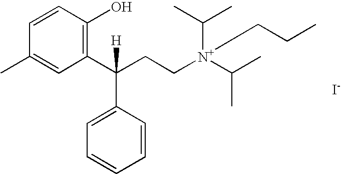

Following the general procedure of EXAMPLE 2 and making non critical variations, but starting with propyl iodide the title compound is obtained.

### Example 6

### (3R)-N-Benzyl-3-(2-hydroxy-5-methylphenyl)-N,N-diisopropyl-3-phenylpropan-1-aminium iodide

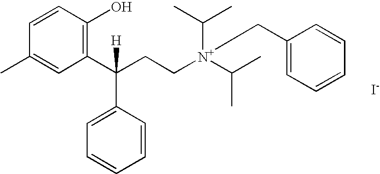

Following the general procedure of EXAMPLE 2 and making non critical variations, but starting with benzyl iodide the title compound is obtained

### Example 7

### (3R)-N-(tert-Butyl)-3-(2-hydroxy-5-methylphenyl)-N,N-dimethyl-3-phenylpropan-1-aminium bromide

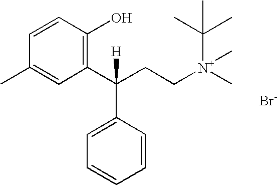

Following the general procedure of EXAMPLE 2 and making non critical variations, but starting with 2-{(1R)-3-[tert-butyl(methyl)amino]-1-phenylpropyl}-4-methylphenol the title compound is obtained.

### Example 8

### (3R)-3-[2-hydroxy-5-(hydroxymethyl)phenyl]-N,N-diisopropyl-N-methyl-3-phenylpropan-1-aminium iodide

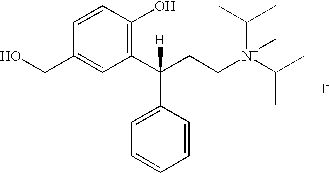

Following the general procedure of EXAMPLE 2 and making non critical variations but starting with 2-[(1R)-3-(diisopropylamino)-1-phenylpropyl]-4-(hydroxymethyl)phenol, the title compound is obtained.

### Example 9

### (3R)-3-(2-hydroxyphenyl)-N,N-diisopropyl-N-methyl-3-phenylpropan-1-aminium bromide

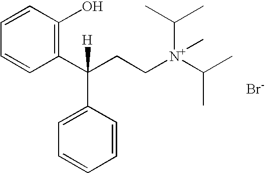

Following the general procedure of EXAMPLE 3 and making non critical variations but starting with 2-[(1R)-3-(diisopropylamino)-1-phenylpropyl]phenol, the title compound is obtained.

### Example 10

### (3S)-3-(2-hydroxyphenyl)-N,N-diisopropyl-N-methyl-3-phenylpropan-1-aminium bromide

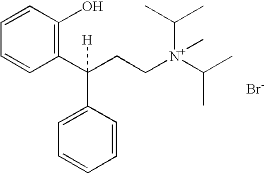

Following the general procedure of EXAMPLES 3 and making non critical variations but starting with 2-[(1S)-3-(diisopropylamino)-1-phenylpropyl]phenol the title compound is obtained.

### Example 11

### (3R)-3-(5-Chloro-2-hydroxyphenyl)-N,N-diisopropyl-N-methyl-3-phenylpropan-1-aminium bromide

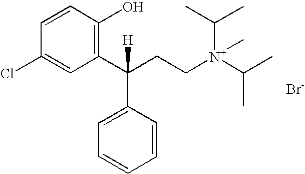

Following the general procedure of EXAMPLE 3 and making non critical variations but starting with 4-chloro-2-[(1R)-3-(diisopropylamino)-1-phenylpropyl]phenol, the title compound is obtained.

### Example 12

### (3R)-3-(5-Bromo-2-hydroxyphenyl)-N,N-diisopropyl-N-methyl-3-phenylpropan-1-aminium bromide

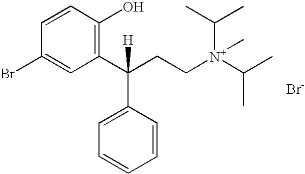

Following the general procedure of EXAMPLE 3 and making non critical variations but starting with 4-bromo-2-[(1R)-3-(diisopropylamino)-1-phenylpropyl]phenol, the title compound is obtained.

### Example 13

### (3R)-3-[2-(acetyloxy)-5-methylphenyl]-N,N-diisopropyl-N-methyl-3-phenylpropan-1-aminium iodide

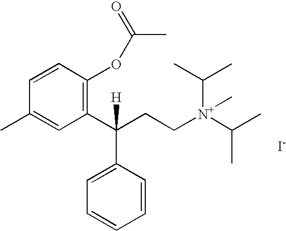

Following the general procedure of EXAMPLE 2 and making non critical variations but starting with 2-[(1R)-3-(diisopropylamino)-1-phenylpropyl]-4-methylphenyl acetate, the title compound is obtained.

### Example 14

### (3R)-3-[2-(isobutyryloxy)-5-methylphenyl]-N,N-diisopropyl-N-methyl-3-phenylpropan-1-aminium iodide

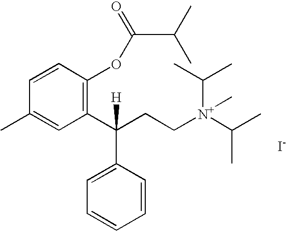

Following the general procedure of EXAMPLE 2 and making non critical variations but starting with 2-[(1R)-3-(diisopropylamino)-1-phenylpropyl]-4-methylphenyl 2-methylpropanoate, the title compound is obtained.

### Example 15

### (3R)-3-(4-Fluorophenyl)-3-(2-hydroxy-5-methylphenyl)-N,N-diisopropyl-N-methylpropan-1-aminium bromide

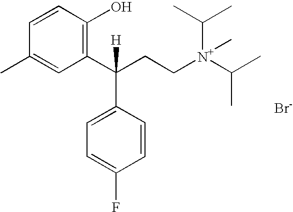

Following the general procedure of EXAMPLE 3 and making non critical variations but starting with 2-[(1R)-3-(diisopropylamino)-1-(4-fluorophenyl)propyl]-4-methylphenol, the title compound is obtained.

### Example 16

### (3R)-3-[2-hydroxy-5-(trifluoromethyl)phenyl]-N,N-diisopropyl-N-methyl-3-phenylpropan-1-aminium bromide

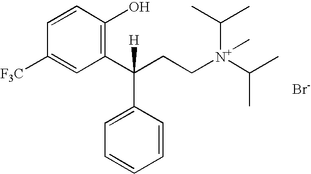

Following the general procedure of EXAMPLE 3 and making non critical variations but starting with 2-[(1R)-3-(diisopropylamino)-1-phenylpropyl]-4-(trifluoromethyl)phenol, the title compound is obtained.

### Example 17

### (3R)-3-[2-(isobutyryloxy)-5-hydroxymethylphenyl]-N,N-diisopropyl-N-methyl-3-phenylpropan-1-aminium bromide

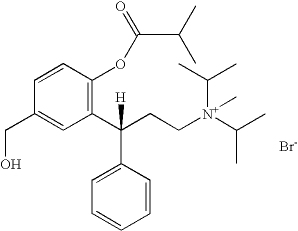

(3R)-3-[2-hydroxy-5-(hydroxymethyl)phenyl]-N,N-diisopropyl-N-methyl-3-phenylpropan-1-aminium bromide is alkylated with isobutyryl bromide to give the title compound.

### Example 18

### (3R)-3-{2-(Acetyloxy)-5-[(acetyloxy)methyl]phenyl}-N,N-diisopropyl-N-methyl-3-phenylpropan-1-aminiumbromide

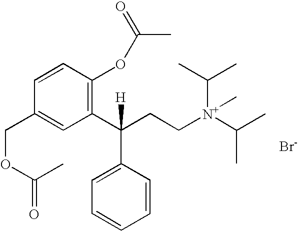

(3R)-3-[2-hydroxy-5-(hydroxymethyl)phenyl]-N,N-diisopropyl-N-methyl-3-phenylpropan-1-aminium bromide is alkylated with acetyl bromide, to give the title compound.

### Example 19

### 2-{(1R)-3-[diisopropyl(methyl)ammonio]-1-phenylpropyl}-4-methylbenzenolate

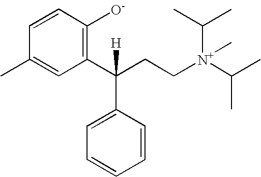

(3R)-3-(2-Hydroxy-5-methylphenyl)-N,N-diisopropyl-N-methyl-3-phenylpropan-1-aminium bromide is passed through an ion exchange column so as to remove the bromide ion and generate the title compound.

Reacting the above compound with an equivalent amount of an acid, for example, methanesulfonic acid, hydrochloric acid, acetic acid, succinic acid generates other salts of the title compound.

Reductive Amination

General Procedure A:

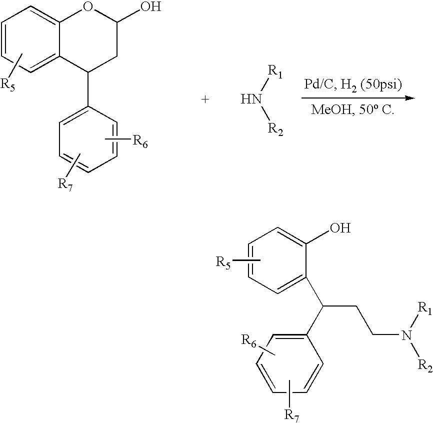

Palladium on activated carbon (1.76 g, 5% wt, Aldrich 20, 568-0) was charged to a hydrogenation vessel under nitrogen followed by a MeOH (20 mL) solution of racemic lactol (4 g, 16.64 mmol) and a secondary amine (42 mmol, 2.5 equiv.). The vessel was filled with hydrogen (50 psi) and the reaction mixture was stirred vigorously at 50° C. overnight. The heterogeneous reaction mixture was filtered through celite. The resulting methanolic solution was concentrated under vacuum.

Cyclic amines, where R₁ and R₂ and the nitrogen form a ring, were obtained in after trituration with hexanes.

General Procedure B:

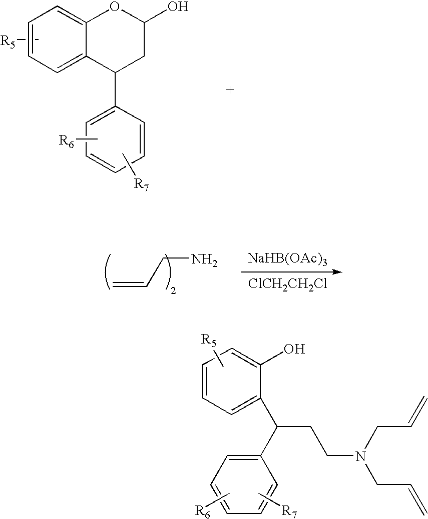

Solid NaHB(OAc)₃ (3 g, 14 mmol) was added under nitrogen to a solution of racemic lactol (2.4 g, 10 mmol) and secondary amine (0.97 g, 1.23 mL, 10 mmol) in 1,2-dichloroethane (35 mL). The reaction mixture was stirred overnight at room temperature. The reaction mixture was quenched with a saturated aqueous solution of NaHCO₃, layers were separated and the aqueous layer was extracted with ether (2×30 mL). The combined organic layers were dried other MgSO₄. After filtration, the solvents were removed under vacuum to give the crude tertiary amine as an oil. The tertiary amine obtained following this procedure was used without purification for the quaternization step.

Quaternization of the Tertiary Amines

General Procedure

Alkyl, benzyl, or allyl including a counter anion such as halide (10 equivalents) were added to a solution of free base of the tertiary amine (0.3 g, 1.02 mmol) in acetone (4 mL). The reaction mixture is stirred overnight at room temperature. The solution is concentrated to initiate the precipitation of the quaternary ammonium salt. The white precipitate is filtered, washed with diethyl ether and dried under vacuum to give the corresponding quaternized salts.

### Example 20

### 1-[3-(2-Hydroxy-5-methylphenyl)-3-phenylpropyl]-1-(2-methylprop-2-enyl)pyrrolidinium Bromide

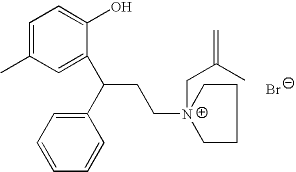

The title compound was produced by reductive amination of 6-methyl-4-phenyl-2-chromanol with pyrrolidine followed by quaternization with prop-2-enyl bromide according to the procedures described above. ¹H NMR (MeOH-d₄): δ 1.90, 2.0-2.25, 2.47-2.71, 3.21-3.31, 3.50-3.64, 3.97, 4.38, 5.36, 5.41, 6.70, 6.88, 6.95, 7.18-7.24, 7.25-7.40.

### Example 21

### 1-[3-(2-Hydroxy-5-methylphenyl)-3-phenylpropyl]-1-(3-methylbut-2-enyl)pyrrolidinium Bromide

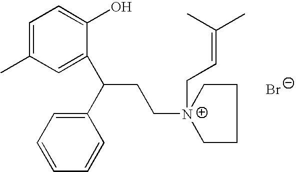

The title compound was produced by reductive amination of 6-methyl-4-phenyl-2-chromanol with pyrrolidine followed by quaternization with 3-methylbut-2-enyl bromide according to the procedures described above. ¹H NMR (MeOH-d₄): δ 1.88, 1.90, 2.0-2.25, 2.40-2.65, 3.18-3.24, 3.38-3.60, 3.97, 4.38, 5.20, 5.41, 6.68, 6.88, 6.95, 7.18-7.24, 7.25-7.40.

### Example 22

### 1-Allyl-1-[3-(2-hydroxy-5-methylphenyl)-3-phenylpropyl]pyrrolidinium Iodide

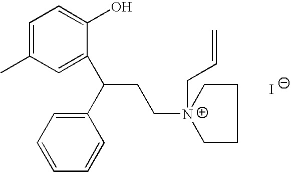

The title compound was produced by reductive amination of 6-methyl-4-phenyl-2-chromanol with pyrrolidine followed by quaternization with allyl iodide according to the procedures described above. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 23

### 1-Allyl-1-[3-(2-hydroxy-5-methylphenyl)-3-phenylpropyl]pyrrolidinium Chloride

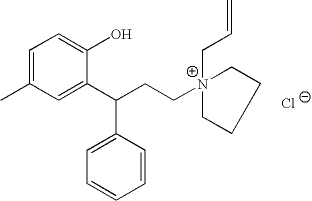

The title compound was produced via an Ion-exchange reaction. The iodide compound of Example 3 (0.6 g) was vigorously stirred with the chloride form of ion-exchange resin AG-2-X8 Bio-Rad (60 g) in 200 mL of an acetonitrile/water mixture (30/70) for 4 h. The resin was filtered on a sintered glass funnel and washed with an acetonitrile/water mixture (30/70) (40 ml). The acetonitrile was removed under vacuum and the remaining water was removed on a lyophilizer to give 0.35 g (72%) of a slightly off-white solid of the titled compound. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 24a

### 3-(2-Hydroxy-5-methylphenyl)-N,N-diallyl-N-methyl-3-phenyl propan-1-aminium Iodide

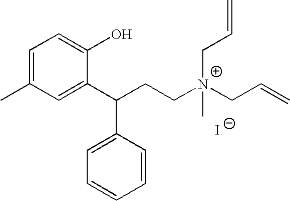

Preparation of 2-[3-(diallylamino)-1-phenylpropyl]-4-methylphenol

The tertiary amine was propuced by reductive amination of the lactol according to the procedures described above.

Preparation of 3-(2-Hydroxy-5-methylphenyl)-N,N-diallyl-N-methyl-3-phenyl propan-1-aminium Iodide

Methyl iodide (2.2 g, 0.96 mL, 0.0155 mol) was added to a solution of the tertiary amine (0.5 g, 1.55 mmol) in a mixture of ether (3 mL) and acetone (1 mL). The reaction mixture was stirred overnight at room temperature to give a white precipitate. The white precipitate was filtered out, triturated with ether, filtered and dried under vacuum to give the title compound. ¹H NMR (MeOH-d₄): δ 2.19, 2.48-2.67, 2.98, 3.1-3.28, 3.96, 4.36, 5.61-5.7, 5.86-6.00, 6.68, 6.84, 7.01, 7.18, 7.29, 7.38.

### Example 24b

### 3-(2-Hydroxy-5-methylphenyl)-N,N-diallyl-N-ethyl-3-phenylpropan-1-aminium Iodide

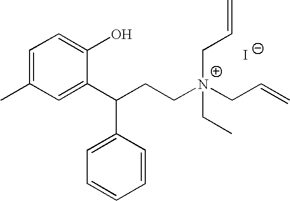

Ethyl iodide (2.42 g, 1.24 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered then washed with ether and dried under vacuum to give the title compound. ¹H NMR (MeOH-d₄): δ 1.25, 2.19, 2.44-2.65, 3.09-3.22, 3.29-3.36, 3.91, 4.35, 5.6-5.7, 5.85-5.99, 6.8, 6.85, 7.0, 7.19, 7.30, 7.39.

### Example 25

### 1-Allyl-1-[3-(2-hydroxy-5-methylphenyl)-3-phenyl propyl]piperidinium Chloride

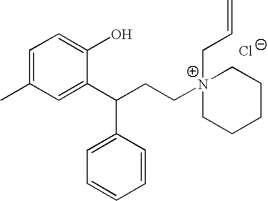

1-[3-(2-hydroxy-5-methylphenyl)-3-phenyl propyl]piperidine was prepared by reductive amination of the lactol with piperidine according to the procedures described above.

Allyl iodide (1.64 g, 0.88 mL, 0.098 mol) was added to a solution of 1-[3-(2-hydroxy-5-methylphenyl)-3-phenyl propyl]piperidine (0.3 g, 0.97 mmol) in a mixture of acetonitrile (6 mL) and methylene chloride (3 mL). The reaction mixture was stirred overnight at room temperature. The solvents were removed under vacuum and the resulting solid triturated with ether to give a solid. The solid was vigorously stirred with the chloride form of ion-exchange resin AG-2-X8 (70 g) in 200 mL of an acetonitrile/water mixture (30/70) for 4 h. The acetonitrile was removed under vacuum and the remaining water removed on a lyophilizer to give the title compound. ¹H NMR (MeOH-d₄): δ 1.64-1.83, 2.19, 2.4-2.59, 3.15-3.33, 4.0, 4.36, 5.56-5.66, 5.76-587, 6.68, 6.85, 7.19, 7.28-7.39.

### Example 26

### 3-(2-Hydroxy-5-methylphenyl)-N,N,N-triallyl-3-phenylpropan-1-aminium Bromide

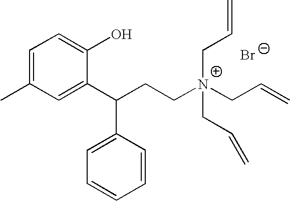

Allyl bromide (1.88 g, 1.34 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered out, washed with ether and dried under vacuum to give the title compound. ¹H NMR (MeOH-d₄): δ 2.18, 2.47-2.67, 3.09-3.26, 3.92, 4.34, 5.64-5.70, 5.9-6.04, 6.68, 6.85, 6.92, 7.20, 7.28-7.37.

### Example 27

### Production of (3S)-3-(2-amino-2-oxo-1,1-diphenylethyl)-1-[2-(2,3-dihydro-1-benzofuran-5-yl)ethyl]-1-methylpyrrolidinium iodide

(3S)-3-(2-amino-2-oxo-1,1-diphenylethyl)-1-[2-(2,3-dihydro-1-benzofuran-5-yl)ethyl]-1-pyrrolidine (1) is prepared according to the procedures described in U.S. Pat. No. 5,096,890. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterized by NMR analysis, mass spectrometry, and melting point determination.

### Example 28

### Production of 4-(diethylmethylaminium)-2-butynyl alpha phenyl cyclohexane glycolate iodide

4-(diethylamino)-2-butynyl alpha phenyl cyclohexane glycolate (1) is prepared according to the procedures described in U.S. Pat. No. 5,973,182. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterized by NMR analysis, mass spectrometry, and melting point determination.

### Example 29

### Production of 3-methyl-3-quinuclidinyl 1-phenyl-2-isoindolinecarboxylate

3-Quinuclidinyl 1-phenyl-2-isoindolinecarboxylate (1) is prepared according to the procedures described in European Patent No.0801067 A1. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterized by NMR analysis, mass spectrometry, and melting point determination.

### Example 30

### Production of (2R)-N-[1-(6-aminopyridin-2-ylmethyl)1-methylpiperdin-4-yl]-2-[(1R)-3,3,-difluorocyclopentyl]-2-hydroxy-2-phenylacetamide iodide.

(2R)-N-[1-(6-aminopyridin-2-ylmethyl)piperdin-4-yl]-2-[(1R)-3,3,difluorocyclopentyl]-2-hydroxy-2-phenylacetamide (1) is prepared according to the procedures described in U.S. Patent Application No. 2001/0051727A1. To COMPOUND (1), free base in toluene, is added methyl iodide (1 ml). Acetonitrile (5 ml) is added to the mixture and stirred over night at 20-25° C. The solvent is removed by blowing dry nitrogen. Acetone (1 ml) and hexane (2 ml) are added and the mixture is filtered at 20-25° C. to give the title compound. The identity of the compound has been further verified and characterized by NMR analysis, mass spectrometry, and melting point determination.

We claim:

1. A method of treating chronic obstructive pulmonary disease (COPD) in a mammal, comprising administering a first pharmaceutical agent and a second pharmaceutical agent, wherein the first pharmaceutical agent comprises a compound of formula I 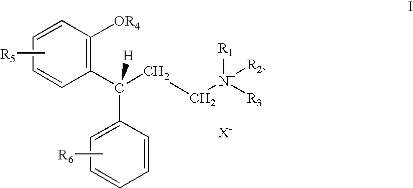
or an enantiomer thereof, wherein:
each R₁, R₂, and R₃ is independently H, C₁-C₅ alkyl optionally substituted with phenyl, or C₂-C₆ alkenyl; or
two of R₁, R₂ and R₃ may form a ring together with the quaternary ammonium nitrogen;
R₄ is
—H,
—CO—R₄₋₁, where R₄₋₁ is
C₁-C₄ alkyl,
C₁-C₄ alkoxy, or
—NR₄₋₂R₄₋₃, where R₄₋₂ and R₄₋₃ are the same or different and are —H or C₁-C₄ alkyl;
R₅ and R₆ are the same or different and are
—H;
C₁-C₄ alkyl optionally substituted with 1 or 2
—OH,
C₁-C₄ alkoxy,
—COOH, or
—CO—O—(C₁-C₃ alkoxy);
—F, —Cl, or Br; or
—CF₃;
where X⁻ is an anion of hydrochloric acid; hydrobromic acid; hydroiodic acid;
sulfuric acid; phosphoric acid; nitric acid; citric acid; methanesulfonic acid; CH₃—(CH₂)ₙ—COOH where n is 0 to 4; HOOC—(CH₂)ₘ—COOH, where m is 1 to 4; HOOC—CH═CH—COOH; or benzoic acid; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.

2. A method of treating chronic obstructive pulmonary disease (COPD) in a mammal, which method comprises administering a first pharmaceutical agent and a second pharmaceutical agent, wherein the first pharmaceutical agent is a compound of formula II 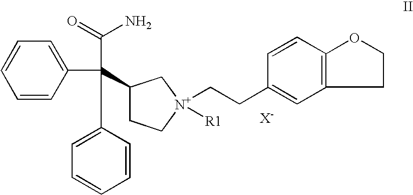
or a stereoisomer thereof, wherein
R₁ is C₁-C₆ alkyl, —CH₂—(C₁-C₄ alkenyl), or —CH₂—(C₁-C₆ alkynyl), each of which is optionally substituted with phenyl, C₁-C₄ alkoxy, or hydroxyl; and
X⁻ is an anion of a pharmaceutically acceptable acid; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.

3. A method of treating chronic obstructive pulmonary disease (COPD) in a mammal, which method comprises administering a first pharmaceutical agent and a second pharmaceutical agent, wherein the first pharmaceutical agent is a compound of formula III 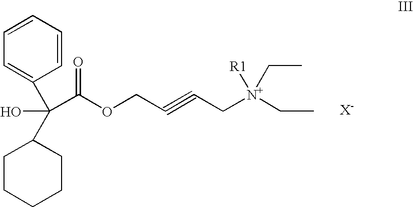
or a stereoisomer thereof, wherein
R₁ is C₁-C₆ alkyl, —CH₂—(C₁-C₄ alkenyl), or —CH₂—(C₁-C₆ alkynyl), each of which is optionally substituted with phenyl, C₁-C₄ alkoxy, or hydroxyl; and
X⁻ is an anion of a pharmaceutically acceptable acid; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.

4. A method of treating chronic obstructive pulmonary disease (COPD) in a mammal, which method comprises administering a first pharmaceutical agent and a second pharmaceutical agent, wherein the first pharmaceutical agent is a compound of formula IV 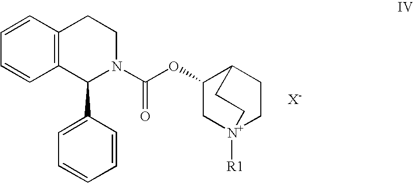
or a stereoisomer thereof, wherein
R₁ is C₁-C₆ alkyl, —CH₂—(C₁-C₄ alkenyl), or —CH₂—(C₁-C₆ alkynyl), each of which is optionally substituted with phenyl, C₁-C₄ alkoxy, or hydroxyl; and
X⁻ is an anion of a pharmaceutically acceptable acid; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.

5. A method of treating chronic obstructive pulmonary disease (COPD) in a mammal, which method comprises administering a first pharmaceutical agent and a second pharmaceutical agent, wherein the first pharmaceutical agent is a compound of formula V 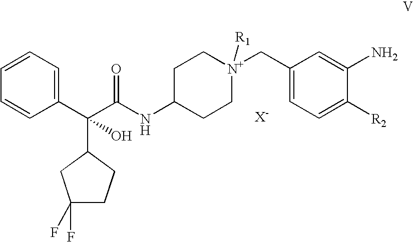
or a stereoisomer thereof, wherein
R₁ is C₁-C₆ alkyl, —CH₂—(C₁-C₄ alkenyl), or —CH₂—(C₁-C₆ alkynyl), each of which is optionally substituted with phenyl, C₁-C₄ alkoxy, or hydroxyl;
R₂ is H or OH; and
X⁻ is an anion of a pharmaceutically acceptable acid; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.

6. The method of claim 2, 3, 4 or 5 wherein X⁻ is an anion of tartaric acid; hydrochloric acid; hydrobromic acid; hydroiodic acid; sulfuric acid; phosphoric acid; nitric acid; citric acid; methanesulfonic acid; CH₃—(CH₂)ₙ—COOH, where n is 0 to 4; HOOC—(CH₂)ₘ—COOH, where m is 1 to 4; HOOC—CH═CH—COOH; or benzoic acid.

7. The method of claim 1, 2, 3, 4 or 5 wherein X⁻ is iodide, bromide, or chloride.

8. The method of claim 1, 2, 3, 4 or 5 comprising administering a pharmaceutical composition of a compound of the formula I, II, III, IV or V.

9. The method of claim 8 wherein the pharmaceutical composition comprises between about 1 mg and about 1000 mg of the compound of the formula I, II, III, IV or V.

10. The method of claim 9 wherein the pharmaceutical composition comprises between about 200 mg and about 800 mg of the compound of the formula I, II, III, IV or V.

11. The method of claim 9 wherein the pharmaceutical composition comprises about 600 mg of the compound of the formula I, II, III, IV or V.

12. The method of claim 1, 2, 3, 4 or 5 wherein the compound of the formula I, II, III, IV or V is administered via inhalation or insufflation.

13. A method of treating chronic obstructive pulmonary disease (COPD) in a mammal, comprising administering a first pharmaceutical agent and a second pharmaceutical agent, wherein the first pharmaceutical agent is
(3R)-3-(2-hydroxy-5-methylphenyl)-N,N-diisopropyl-N-methyl-3-phenylpropan-1-aminium iodide;
(3R)-3-(2-hydroxy-5-methylphenyl)-N,N-diisopropyl-N-methyl-3-phenylpropan-1-aminium bromide;
(3R)-N-ethyl-3-(2-hydroxy-5-methylphenyl)-N,N-diisopropyl-3-phenylpropan-1-aminium iodide;
(3R)-3-(2-hydroxy-5-methylphenyl)-N,N-diisopropyl-3-phenyl-N-propylpropan-1-aminium iodide;
(3R)-N-benzyl-3-(2-hydroxy-5-methylphenyl)-N,N-diisopropyl-3-phenylpropan-1-aminium iodide;
(3R)-N-(tert-butyl)-3-(2-hydroxy-5-methylphenyl)-N,N-dimethyl-3-phenylpropan-1-aminium bromide;
(3R)-3-[2-hydroxy-5-(hydroxymethyl)phenyl]-N,N-diisopropyl-N-methyl-3-phenylpropan-1-aminium iodide;
(3R)-3-(2-hydroxyphenyl)-N,N-diisopropyl-N-methyl-3-phenylpropan-1-aminium bromide;
(3S)-3-(2-hydroxyphenyl)-N,N-diisopropyl-N-methyl-3-phenylpropan-1-aminium bromide;
(3R)-3-(5-chloro-2-hydroxyphenyl)-N,N-diisopropyl-N-methyl-3-phenylpropan-1-aminium bromide;
(3R)-3-(5-bromo-2-hydroxyphenyl)-N,N-diisopropyl-N-methyl-3-phenylpropan-1-aminium bromide;
(3R)-3-[2-(acetyloxy)-5-methylphenyl]-N,N-diisopropyl-N-methyl-3-phenylpropan-1-aminium iodide;
(3R)-3-[2-(isobutyryloxy)-5-methylphenyl]-N,N-diisopropyl-N-methyl-3-phenylpropan-1-aminium iodide;
(3R)-3-(4-fluorophenyl)-3-(2-hydroxy-5-methylphenyl)-N,N-diisopropyl-N-methylpropan-1-aminium bromide;
(3R)-3-[2-hydroxy-5-(trifluoromethyl)phenyl]-N,N-diisopropyl-N-methyl-3-phenylpropan-1-aminium bromide;
(3R)-3-[2-(isobutyryloxy)-5-hydroxymethylphenyl]-N,N-diisopropyl-N-methyl-3-phenylpropan-1-aminium bromide;
(3R)-3-{2-(acetyloxy)-5-[(acetyloxy)methyl]phenyl}-N,N-diisopropyl-N-methyl-3-phenylpropan-1-aminiumbromide;
2-{(1R)-3-[diisopropyl(methyl)ammonio]-1-phenylpropyl}-4-methylbenzenolate;
1-[3-(2-hydroxy-5-methylphenyl)-3-phenylpropyl]-1-(2-methylprop-2-enyl)pyrrolidinium bromide;
1-[3-(2-hydroxy-5-methylphenyl)-3-phenylpropyl]-1-(3-methylbut-2-enyl)pyrrolidinium bromide;
1-allyl-1-[3-(2-hydroxy-5-methylphenyl)-3-phenylpropyl]pyrrolidinium iodide;
1-allyl-1-[3-(2-hydroxy-5-methylphenyl)-3-phenylpropyl]pyrrolidinium chloride;
3-(2-hydroxy-5-methylphenyl)-N,N-diallyl-N-methyl-3-phenyl propan-1-aminium iodide;
3-(2-hydroxy-5-methylphenyl)-N,N-diallyl-N-ethyl-3-phenylpropan-1-aminium iodide;
1-allyl-1-[3-(2-hydroxy-5-methylphenyl)-3-phenyl propyl]piperidinium chloride;
3-(2-hydroxy-5-methylphenyl)-N,N,N-triallyl-3-phenylpropan-1-aminium bromide;
(3S)-3-(2-amino-2-oxo-1,1-diphenylethyl)-1-[2-(2,3-dihydro-1-benzofuran-5-yl)ethyl]-1-methylpyrrolidinium iodide;
4-(diethylmethylaminium)-2-butynyl alpha phenyl cyclohexane glycolate iodide;
3-methyl-3-quinuclindinyl 1-phenyl-2-isoindolinecarboxylate; or
(2R)-N-[1-(6-aminopyridin-2-ylmethyl) 1-methylpiperdin-4-yl]-2-[(1R)-3,3,-difluorocyclopentyl]-2-hydroxy-2-phenylacetamide iodide; and
the second pharmaceutical agent is an Adenosine A₂ₐ Receptor Agonist, a D2-Dopamine Receptor Agonist, a PDE Inhibitor, a corticosteroid, a norepinephrine reuptake inhibitor, or 4-hydroxy-7-[2-[2-[3-[2-phenylethoxy]-propylsulphonyl]ethylamino]ethyl]-1,3-benzothiazol-2(3H)-one.
